(19) United States
(12) Patent Application Publication
(10) Pub. No.: US 2020/0166740 A1
(43) Pub. Date: May 28, 2020

(54) LINEAR ACTUATOR FOR ENDOSCOPE, OPTICAL UNIT FOR ENDOSCOPE, AND ENDOSCOPE

(71) Applicant: OLYMPUS CORPORATION, Tokyo (JP)

(72) Inventor: Hiroyuki NAGAMIZU, Sagamihara-shi (JP)

(73) Assignee: OLYMPUS CORPORATION, Tokyo (JP)

(21) Appl. No.: 16/776,731

(22) Filed: Jan. 30, 2020

Related U.S. Application Data
(63) Continuation of application No. PCT/JP2018/023231, filed on Jun. 19, 2018.

(30) Foreign Application Priority Data
Aug. 4, 2017 (JP) 2017-151564

(51) Int. Cl. G02B 23/24; A61B 1/00; H01F 7/16; G02B 7/10

(52) U.S. Cl. CPC G02B 23/2438; A61B 1/00096; G02B 7/10; H01F 7/1615; G02B 23/2476; A61B 1/00188

(57) ABSTRACT

A linear actuator for endoscope includes: a movable frame made of a magnetic material; a fixed frame in which the movable frame is provided so as to be movable forward and backward, the fixed frame being made of a non-magnetic material; two coils formed by being wound on an outer surface of the fixed frame, the two coils being disposed side by side in a longitudinal axis direction; a permanent magnet disposed in a manner stacked and superimposed on the two coils; and two yokes respectively disposed at a front and a back of the permanent magnet and the two coils.

### CROSS REFERENCE TO RELATED APPLICATION

This application is a continuation application of PCT/JP2018/023231 filed on Jun. 19, 2018 and claims benefit of Japanese Application No. 2017-151564 filed in Japan on Aug. 4, 2017, the entire contents of which are incorporated herein by this reference.

### BACKGROUND OF INVENTION

### 1. Field of the Invention

The present invention relates to a linear actuator for endoscope, which includes an electromagnetic coil and a magnet, an optical unit for endoscope, and an endoscope.

### 2. Description of the Related Art

Conventionally, endoscopes, which are configured to be capable of observing organs in a body cavity or an inside of an engine by inserting an elongated insertion portion into a body cavity or an engine plant, have been widely used.

Such endoscopes include an optical unit including an objective optical system. An optical unit provided in endoscopes is mostly provided at a distal end portion of an insertion portion. Such an optical unit includes a mobile frame that holds an optical element and is movable in an optical axis direction for a zooming function or a focusing function. As a technique of such a movable frame, for example, the technique recited in the United States Patent Publication No. 2010-0127580 is known, in which the movable frame is driven to move forward and backward by a linear actuator including an electromagnetic coil and a magnet.

### SUMMARY OF THE INVENTION

A linear actuator for endoscope according to an aspect of the present invention includes: a movable frame made of a magnetic material, the movable frame holding a lens; a fixed frame in which the movable frame is provided so as to be movable forward and backward, the fixed frame being made of a non-magnetic material; two coils formed by being wound on an outer surface of the fixed frame, the two coils being disposed side by side in a longitudinal axis direction; a permanent magnet disposed in a manner stacked and superimposed on the two coils; and two yokes respectively disposed at a front and a back of the permanent magnet and the two coils.

An optical unit for endoscope according to an aspect of the present invention includes a linear actuator for endoscope. The linear actuator includes: a movable frame made of a magnetic material, the movable frame holding a lens; a fixed frame in which the movable frame is provided so as to be movable forward and backward, the fixed frame being made of a non-magnetic material; two coils formed by being wound on an outer surface of the fixed frame, the two coils being disposed side by side in a longitudinal axis direction; a permanent magnet disposed in a manner stacked and superimposed on the two coils; and two yokes respectively disposed at a front and a back of the permanent magnet and the two coils.

An endoscope according to an aspect of the present invention includes an insertion portion having a distal end portion in which an optical unit for endoscope is provided. The optical unit for endoscope includes a linear actuator for endoscope, and the linear actuator includes: a movable frame made of a magnetic material, the movable frame holding a lens; a fixed frame in which the movable frame is provided so as to be movable forward and backward, the fixed frame being made of a non-magnetic material; two coils formed by being wound on an outer surface of the fixed frame, the two coils being disposed side by side in a longitudinal axis direction; a permanent magnet disposed in a manner stacked and superimposed on the two coils; and two yokes respectively disposed at a front and a back of the permanent magnet and the two coils.

### BRIEF DESCRIPTION OF THE DRAWINGS

FIG. 1 is a perspective view illustrating an overall configuration of an endoscope.

FIG. 2 schematically illustrates an image pickup apparatus and a control section.

FIG. 3 is an exploded perspective view illustrating a movable lens unit.

FIG. 4 is a perspective view illustrating an appearance of the movable lens unit.

FIG. 5 is a sectional view illustrating the movable lens unit.

FIG. 6 schematically illustrates two coils and the control section.

FIG. 7 is a sectional view illustrating the movable lens unit, with a movable frame moved forward.

FIG. 8 is a sectional view illustrating the movable lens unit, with the movable frame moved backward.

FIG. 9 schematically illustrates two coils and a control section according to a first modified example.

FIG. 10 is a sectional view illustrating a movable lens unit, with a movable frame moved forward, according to the first modified example.

FIG. 11 is a sectional view illustrating the movable lens unit, with the movable frame moved backward, according to the first modified example.

FIG. 12 is a perspective view illustrating an appearance of a movable lens unit according to a second modified example.

FIG. 13 is a sectional view illustrating the movable lens unit according to the second modified example.

FIG. 14 is a perspective view illustrating an appearance of a movable lens unit according to a third modified example.

FIG. 15 is a sectional view illustrating the movable lens unit according to the third modified example.

FIG. 16 is a perspective view illustrating an appearance of a movable lens unit according to a fourth modified example.

FIG. 17 is a sectional view illustrating the movable lens unit according to the fourth modified example.

FIG. 18 is a perspective view illustrating an appearance of a movable lens unit according to a fifth modified example.

FIG. 19 is a sectional view illustrating the movable lens unit according to the fifth modified example.

### DETAILED DESCRIPTION OF THE PREFERRED EMBODIMENT(S)

Hereinafter a preferred embodiment of the present invention will be described with reference to drawings.

Note that a different scale size is used for each of the components in order to allow each of the components to be illustrated in a recognizable size in the drawings to be used for the description below, and the present invention is not limited to the number, shapes, ratio of the sizes of the components, and a relative positional relationship among the components shown in these drawings. In addition, in the description below, there is a case where the up and down directions viewing toward the paper surface of the drawings are referred to as the upper portion and the lower portion of a component.

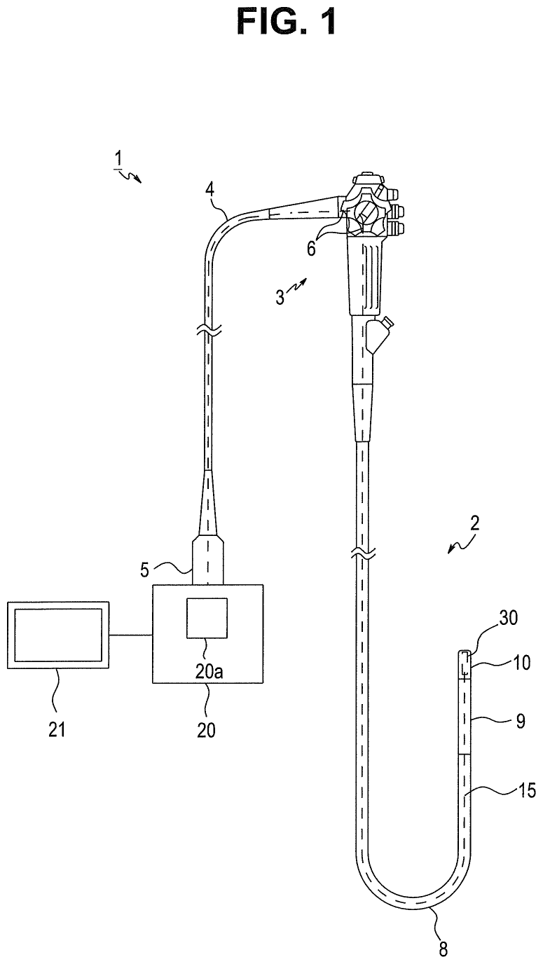

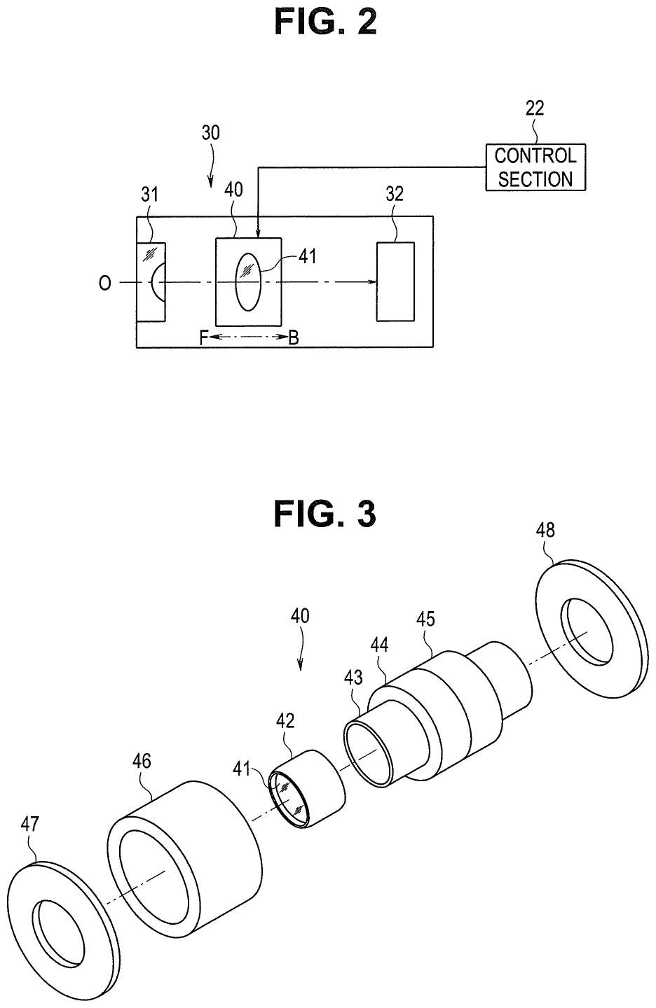

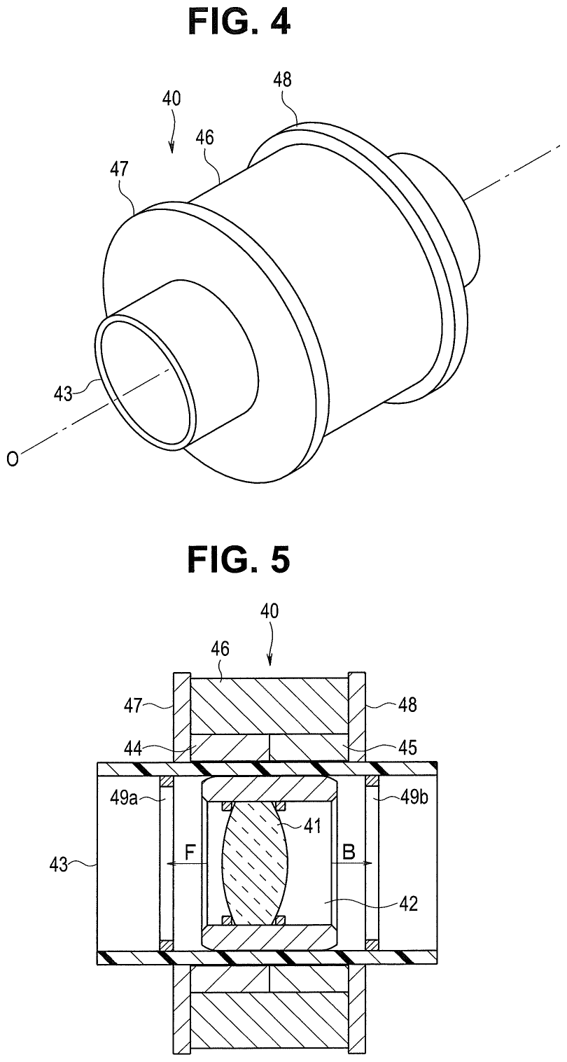

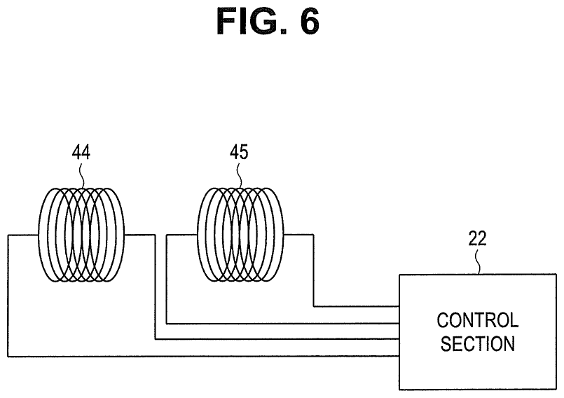

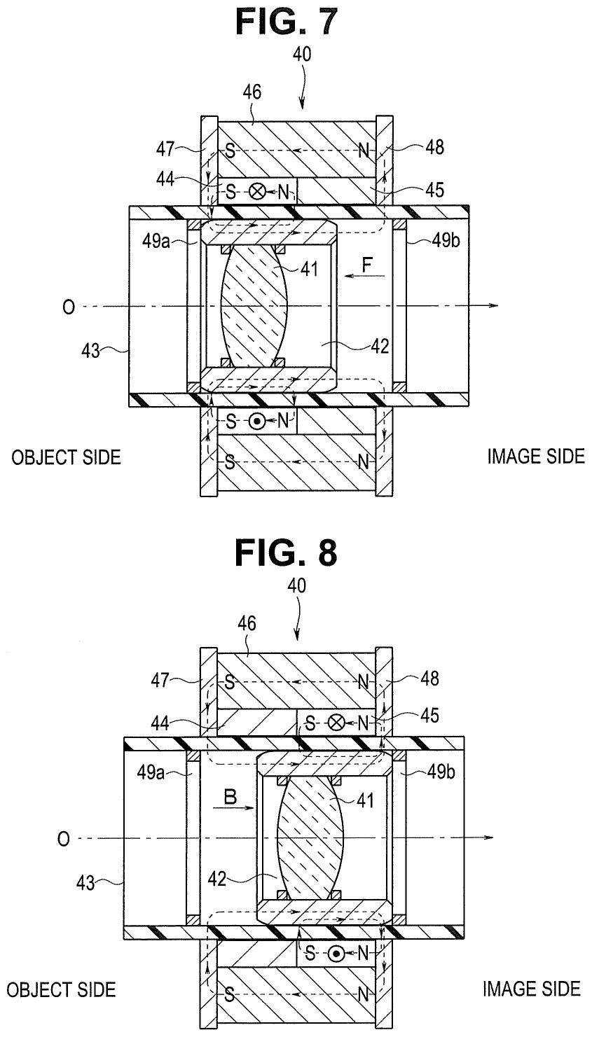

FIG. 1 is a perspective view illustrating an overall configuration of an endoscope, FIG. 2 schematically illustrates an image pickup apparatus and a control section, FIG. 3 is an exploded perspective view illustrating a movable lens unit, FIG. 4 is a perspective view illustrating an appearance of the movable lens unit, FIG. 5 is a sectional view illustrating the movable lens unit, FIG. 6 schematically illustrates the two coils and the control section, FIG. 7 is a sectional view illustrating the movable lens unit, with the movable frame moved forward, and FIG. 8 is a sectional view illustrating the movable lens unit, with the movable frame moved backward.

First, one example of a configuration of an endoscope according to the present invention will be described with reference to FIG. 1.

An endoscope 1, which is an endoscope system according to the present embodiment, is configured to be introducible into a subject such as a human body and configured to optically pick up an image of a predetermined observation site in the subject.

Note that the subject into which the endoscope 1 is introduced is not limited to a human body, but may be another living body, or artificial object such as a machine or a structure.

The endoscope 1 mainly includes an insertion portion 2 configured to be introduced into a subject, an operation portion 3 located at the proximal end of the insertion portion 2, and a universal cord 4 extended from a side portion of the operation portion 3.

The insertion portion 2 includes a distal end portion 10 disposed at the distal end of the insertion portion, a bendable bending portion 9 disposed on the proximal end side of the distal end portion 10, and a flexible tube portion 8 having flexibility that is disposed on the proximal end side of the bending portion 9 and connected to the distal end side of the operation portion 3.

Note that the endoscope 1 may be what is called a rigid endoscope which does not include a flexible part at the insertion portion 2.

The distal end portion 10 includes an image pickup apparatus 30 which is an optical unit for endoscope in which an image pickup module is incorporated. In addition, the operation portion 3 is provided with an angle operation knob 6 for operating the bending of the bending portion 9.

The universal cord 4 includes, at the proximal end portion thereof, an endoscope connector 5 configured to be connected to an external apparatus 20. The external apparatus 20 to which the endoscope connector 5 is connected is connected to an image display section 21 such as a monitor, through a cable.

In addition, the endoscope 1 includes a composite cable 15 inserted through the universal cord 4, the operation portion 3, and the insertion portion 2, and an optical fiber bundle (not illustrated) that transmits illumination light from a light source section disposed in the external apparatus 20.

The composite cable 15 is configured to electrically connect the endoscope connector 5 and the image pickup apparatus 30. The endoscope connector 5 is connected to the external apparatus 20, thereby causing the image pickup apparatus 30 to be electrically connected to the external apparatus 20 through the composite cable 15.

Current supply from the external apparatus 20 to the image pickup apparatus 30 and communication between the external apparatus 20 and the image pickup apparatus 30 are performed through the composite cable 15.

The external apparatus 20 is provided with an image processing section 20a. The image processing section 20a generates a video signal based on an image pickup device output signal outputted from the image pickup apparatus 30, and outputs the generated video signal to the image display section 21. That is, according to the present embodiment, an optical image (endoscopic image) obtained by image pickup with the image pickup apparatus 30 is displayed as video on the image display section 21.

Note that the endoscope 1 is not limited to the configuration in which the endoscope 1 is connected to the external apparatus 20 or the image display section 21, but the endoscope 1 may include a part of or an entirety of the image processing section or the monitor, for example.

In addition, the optical fiber bundle is configured to transmit the light emitted from the light source section of the external apparatus 20 to an illumination window as an illumination light emission portion of the distal end portion 10. Furthermore, the light source section may be disposed in the operation portion 3 or the distal end portion 10 of the endoscope 1.

As illustrated in FIG. 2, the image pickup apparatus 30 as an optical unit for endoscope includes an objective optical system 31, a movable lens unit 40 that configures a linear actuator for endoscope, and a solid-state image pickup device 32.

Although only one objective optical system 31 is illustrated here, a plurality of lens groups may be provided.

The movable lens unit 40 is provided with a linear actuator configured to drive a movable lens 41, to be described later, and move the movable lens forward and backward (F-B direction) along a photographing optical axis O. Note that the movable lens unit 40 is configured such that the linear actuator that drives the movable lens 41 to move the movable lens forward and backward is driven and controlled by the control section 22.

The solid-state image pickup device 32 is an extremely small electronic component in which a plurality of elements configured to output an electric signal corresponding to the incident light at a predetermined timing are aligned on a planar light-receiving portion, and a type generally called as CCD (charge coupled device) or CMOS (complementary metal oxide semiconductor) sensor, or other various types of devices are applied, for example.

As shown in FIGS. 3 to 5, the movable lens unit 40 includes: the movable lens 41; a movable frame 42 that holds the movable lens 41; a cylindrical fixed frame 43; two coils, i.e., a first coil 44 and a second coil 45 formed by winding a metal wire made of copper, or the like, on the outer circumference (outer surface) of the fixed frame 43; a cylindrical permanent magnet 46 disposed so as to cover the entire circumferences of the two coils, i.e., the first coil 44 and the second coil 45; and doughnut-like disk-shaped yokes 47, 48 disposed so as to sandwich the first coil 44, the second coil 45, and the permanent magnet 46 in the front-back direction. The movable lens unit 40 is configured by assembling the various components as illustrated in FIG. 4.

Note that stoppers 49a, 49b are disposed inside the fixed frame 43. As illustrated in FIG. 5, the stoppers 49a, 49b are configured to respectively contact the end surfaces of the movable frame 42 that holds the movable lens 41, to restrict the forward and backward movement of the movable frame 42.

The image pickup optical system including the objective optical system 31 and the movable lens 41 forms an optical image of an object. Note that the image pickup optical system is not limited to the optical system in which the objective optical system 31 and the movable lens 41 are separated from each other. The entirety of the image pickup optical system may be disposed in the movable lens unit 40.

The movable frame 42 and the yokes 47, 48 are made of a magnetic material. The fixed frame 43 is made of a non-magnetic material. Since the yokes 47, 48 are made of the magnetic material, an electromagnet is configured by the first coil 44 and the second coil 45 sandwiched by the yokes 47, 48 disposed respectively on the front side of the first coil and the back side of the second coil. Note that the image pickup optical system forms the optical image of the object on the light-receiving portion.

The first coil 44 and the second coil 45 are formed by being wound on the outer circumferential surface of the fixed frame 43 such that the winding directions are set in the same direction and the first coil and the second coil are disposed adjacently side by side in the longitudinal axis direction along the photographing optical axis O, as illustrated in FIG. 5.

The permanent magnet 46 is disposed in a manner stacked and superimposed on the first coil 44 and second coil 45. In the present embodiment, the distal end side, which is the front side, of the permanent magnet 46 is magnetized to be the S-pole, and the proximal end side, which is the back side, of the permanent magnet 46 is magnetized to be the N-pole.

The winding direction of each of the first coil 44 and the second coil 45 is specified such that the distal end side, which is the front side, of each of the coils is magnetized to be the S-pole, and the proximal end side, which is the back side, of each of the coils is magnetized to be the N-Pole when current is supplied to each of the coils.

Note that the first coil 44 and the second coil 45 in the present embodiment individually receive current from the control section 22, as illustrated in FIG. 6. That is, the first coil 44 and the second coil 45 receive current supply from the control section 22 through the electric circuits of the two individual systems.

When the movable lens unit 40 configured as described above is energized by the current being supplied from the control section 22 to the first coil 44, the first coil 44 is magnetized as illustrated in FIG. 7, thereby causing the magnetic force of the front-side yoke 47 to increase. Note that the magnetic force of the back-side yoke 48 at this time remains substantially unchanged, since the yoke 48 is positioned away from the first coil 44.

In this case, the movable frame 42 made of the magnetic material is drawn by the attracting force from the front-side yoke 47 whose magnetic force has been increased, and the movable frame moves forward (direction of the arrow F) of the object side. Then, the movable frame 42 stops by the front end thereof contacting the stopper 49a.

On the other hand, when the movable lens unit 40 is energized by the current being supplied from the control section 22 to the second coil 45, the second coil 45 is magnetized as illustrated in FIG. 8, thereby causing the magnetic force of the back-side yoke 48 to increase. Note that the magnetic force of the front-side yoke 47 at this time remains substantially unchanged, since the yoke 47 is positioned away from the second coil 45.

In this case, the movable frame 42 made of the magnetic material is drawn by the attracting force from the back-side yoke 48 whose magnetic force has been increased, and the movable frame moves backward (direction of the arrow B) of the image side. Then, the movable frame 42 stops by the back end thereof contacting the stopper 49b.

As described above, the movable lens unit 40 which is the linear actuator for endoscope according to the present embodiment drives the movable frame 42 that holds the movable lens 41 to move the movable frame forward or backward (F-B direction) by the energization being switched between the two coils, i.e., the first coil 44 and the second coil 45.

Note that the movable lens unit 40 includes the one permanent magnet 46 disposed so as to be stacked on the outside of the first coil 44 and the second coil 45 that are disposed side by side in the axis direction, which enables shortening of the length of the movable lens unit 40 in the axis direction along the photographing optical axis O. Such a configuration also enables shortening of the length of the image pickup apparatus 30 which is the optical unit for endoscope.

Furthermore, the endoscope 1 includes the image pickup apparatus 30 incorporated in the rigid distal end portion 10 of the insertion portion 2, which also enables shortening of the length of the distal end portion 10.

The movable lens unit 40 does not include a plurality of permanent magnets but includes one permanent magnet 46. As a result, repulsion caused by the repulsive force of magnets is not generated, which enables the permanent magnet 46 to be disposed easily and provides a configuration enabling easy assembly compared with the conventional configuration.

As described above, it is possible to configure the movable lens unit 40 as the linear actuator for endoscope, a size reduction of which is enabled by the shortening of the length and assembling performance of which is improved, compared with the conventional unit. In addition, similarly, the size reduction by the shortening of the length can be enabled for the image pickup apparatus 30, as the optical unit for endoscope, in which the movable lens unit 40 is incorporated, and the distal end portion 10 of the endoscope 1 in which the image pickup apparatus 30 is incorporated.

Note that the movable lens unit 40 is configured to cause the movable lens 41 to move forward and backward for the zooming function for switching between the near point observation and the far point observation or the focusing function for focus adjustment.

The configuration of the linear actuator included in the movable lens unit 40 can be diverted to the functions of the endoscope, for example, a function for pulling and relaxing the bending operation wire for bending the bending portion 9 and a function for pulling and relaxing the operation wire for causing the treatment instrument raising stand, which is provided at the distal end portion 10 of the lateral-view/oblique-view endoscope, to rise and fall.

### MODIFIED EXAMPLES

The movable lens unit 40, which is a linear actuator for endoscope, may be configured as shown in various modified examples below.

### First Modified Example

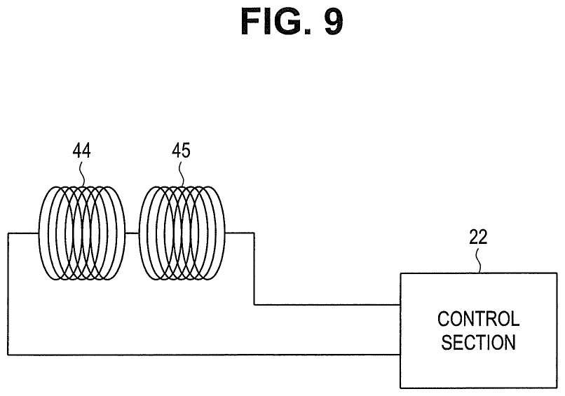

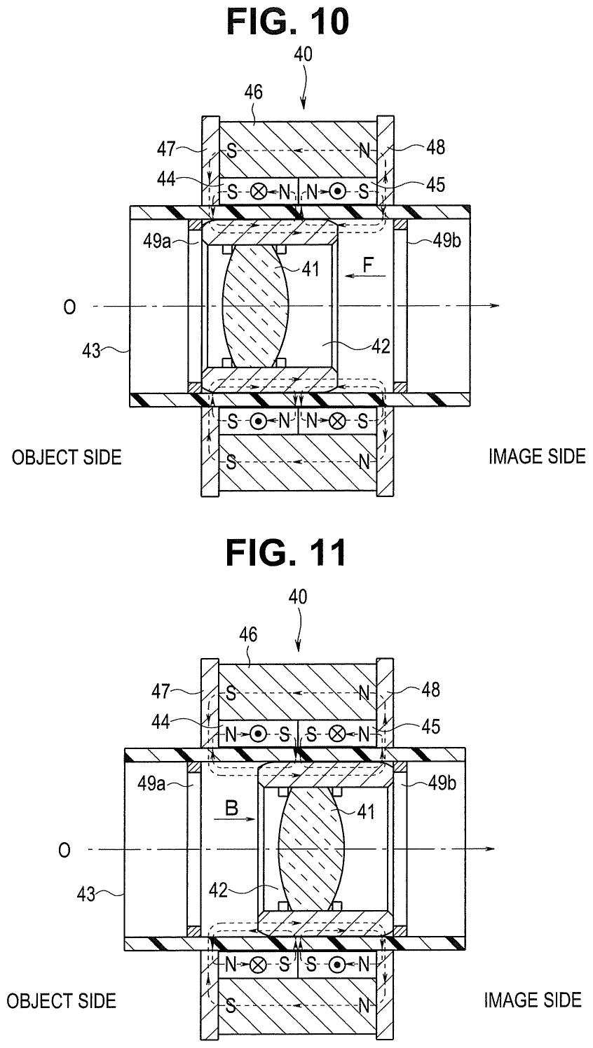

FIG. 9 schematically illustrates two coils and a control section according to a first modified example, FIG. 10 is a sectional view illustrating a movable lens unit, with a movable frame moved forward, according to the first modified example, and FIG. 11 is a sectional view illustrating the movable lens unit, with the movable frame moved backward, according to the first modified example.

The first coil 44 and the second coil 45 in the present modified example receive current from the control section 22 through an electric circuit of a single system, which is connected in series as illustrated in FIG. 9. The first coil 44 and the second coil 45 are formed by being wound such that the wound directions are set in the directions opposite to each other, and are disposed side by side on the outer circumferential surface of the fixed frame 43.

In addition, since the winding directions of the first coil 44 and the second coil 45 are opposite to each other, when current in a predetermined direction is supplied to the first and second coils 44, 45, the coils 44 and 45 are magnetized so as to have reverse N-S polarity at the distal end sides which are the front sides of the respective coils and at the proximal end sides which are the back sides of the respective coils.

When the movable lens unit 40 configured as described above is energized by the current in a predetermined one direction being supplied from the control section 22 to the first coil 44 and the second coil 45, the first coil 44 is magnetized to have the same polarity as that of the permanent magnet 46 and the second coil is magnetized to have the reverse polarity to that of the permanent magnet 46, as illustrated in FIG. 10.

This causes the magnetic force of the front-side yoke 47 to increase to cancel the magnetic force on the back side of the permanent magnet 46, which causes the magnetic force of the back-side yoke 48 to decrease.

As a result, the movable frame 42 made of a magnetic material is drawn to the front-side yoke 47 due to the decrease of the magnetic force of the back-side yoke 48 in addition to the attractive force from the front-side yoke 47 whose magnetic force has been increased, and the movable frame moves forward (direction of the arrow F) of the object side. Then, the movable frame 42 stops by the front end thereof contacting the stopper 49a.

On the other hand, when the movable lens unit 40 is energized by the current in another direction opposite to the predetermined one direction being supplied from the control section 22 to the first coil 44 and the second coil 45, the first coil 44 is magnetized to have the reverse polarity to that of the permanent magnet 46 and the second coil is magnetized to have the same polarity as that of the permanent magnet 46, as illustrated in FIG. 11.

Therefore, the magnetic force on the front side of the permanent magnet 46 is canceled, thereby causing the magnetic force of the front-side yoke 47 to decrease, and causing the magnetic force on the back-side yoke 48 to increase.

As a result, the movable frame 42 made of the magnetic material is drawn to the back-side yoke 48 due to the decrease of the magnetic force of the front-side yoke 47 in addition to the attractive force from the back-side yoke 48 whose magnetic force has been increased, and the movable frame moves backward (direction of the arrow B) of the image side. Then, the movable frame 42 stops by the back side thereof contacting the stopper 49b.

As described above, the movable lens unit 40 which is the linear actuator for endoscope according to the present modified example drives the movable frame 42 that holds the movable lens 41 to move the movable frame forward or backward (F-B direction) by the direction of energization to the first coil 44 and the second coil 45 being switched.

With such a configuration, the movable lens unit 40 is capable of obtaining the above-described operational effects, and is capable of surely moving the movable frame 42 that holds the movable lens 41 forward and backward.

Note that, when the first coil 44 and the second coil 45 are configured to receive current independently from the control section 22 through different circuits, as shown in FIG. 6, the energization may be controlled by the control section 22 such that the first coil 44 is magnetized to have the same polarity as that of the permanent magnet 46 and the second coil is magnetized to have the reverse polarity to that of the permanent magnet 46, thereby causing the movable frame 42 to move forward of the object side.

When moving the movable frame 42 backward of the image side, the energization is controlled by the control section 22 such that the first coil 44 is magnetized to have the reverse polarity to that of the permanent magnet 46 and the second coil is magnetized to have the same polarity as that of the permanent magnet 46.

### Second Modified Example

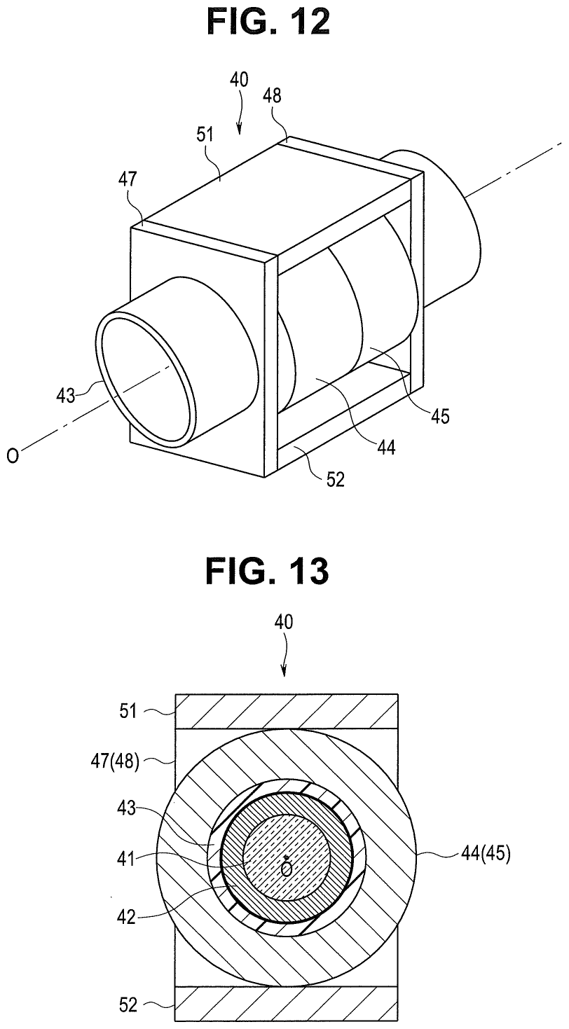

FIG. 12 is a perspective view illustrating an appearance of a movable lens unit according to the second modified example, and FIG. 13 is a sectional view illustrating the movable lens unit according to the second modified example.

As illustrated in FIG. 12, the movable lens unit 40 may include planar permanent magnets 51, 52 stacked in the up/down direction of the first coil 44 and the second coil 45, in the present modified example. That is, the permanent magnet 51 and the permanent magnet 52 are disposed line-symmetrically, with the optical axis O as a symmetry axis. Note that the yokes 47, 48 in the present modified example have rectangular outer shapes in accordance with the shapes of the permanent magnets 51, 52.

Other configurations are the same as those in the above-described embodiment. Such a configuration enables further size reduction of the movable lens unit 40.

### Third Modified Example

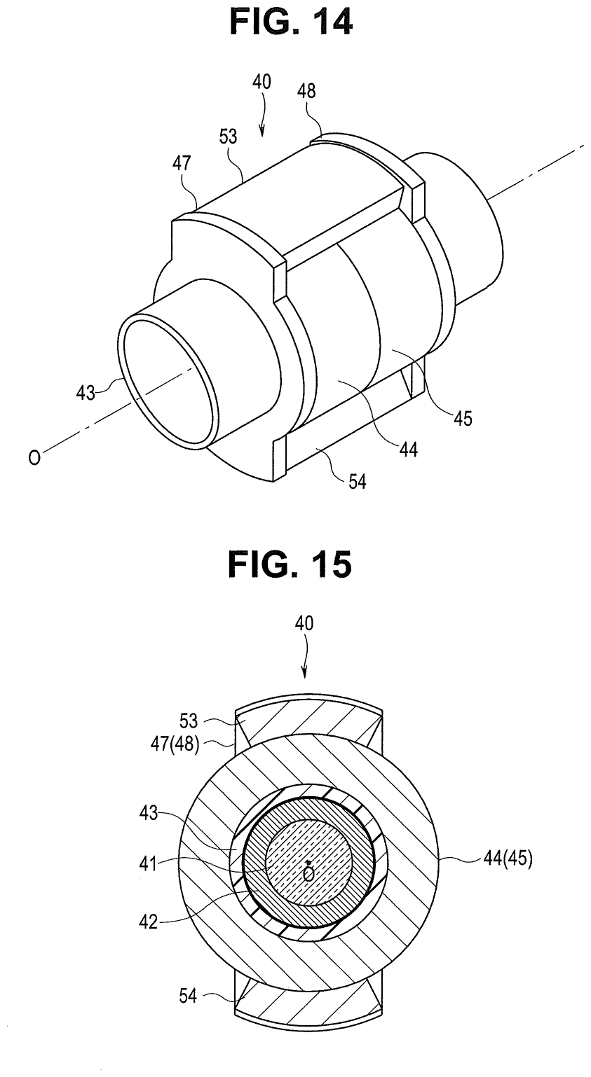

FIG. 14 is a perspective view illustrating an appearance of a movable lens unit according to the third modified example, and FIG. 15 is a sectional view illustrating the movable lens unit according to the third modified example.

As illustrated in FIGS. 14 and 15, the movable lens unit 40 may include two permanent magnets 53, 54, each of which has an arcuate cross-section, stacked in the up/down direction of the first coil 44 and the second coil 45, in the present modified example. That is, the permanent magnet 51 and the permanent magnet 52 are disposed line-symmetrically, with the optical axis O as a symmetry axis. Note that also the yokes 47, 48 in the present modified example have shapes in accordance with the shapes of the permanent magnets 51, 52.

Other configurations are the same as those in the above-described embodiment. Also such a configuration enables further size reduction of the movable lens unit 40.

### Fourth Modified Example

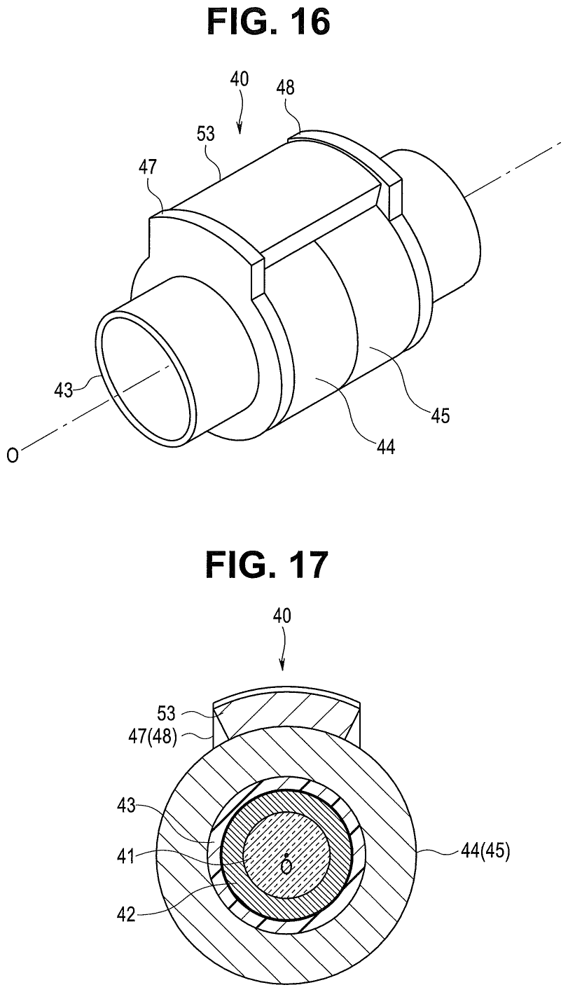

FIG. 16 is a perspective view illustrating an appearance of a movable lens unit according to the fourth modified example, and FIG. 17 is a sectional view illustrating the movable lens unit according to the fourth modified example.

As illustrated in FIGS. 16 and 17, the movable lens unit 40 may include one permanent magnet 53 having an arcuate cross-section and stacked on the upper portion of the first coil 44 and the second coil 45.

Other configurations are the same as those in the above-described embodiment. Also such a configuration enables further size reduction of the movable lens unit 40.

### Fifth Modified Example

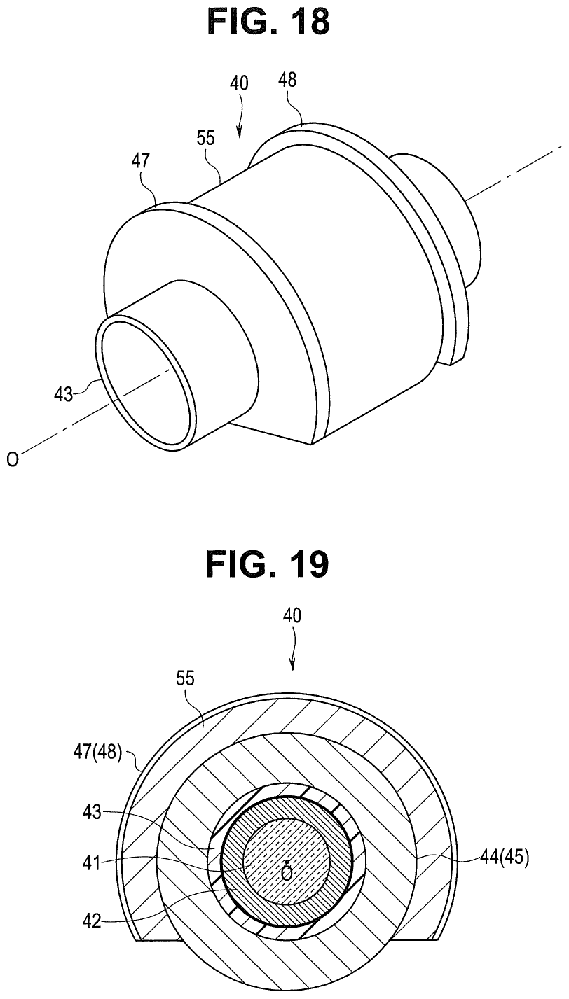

FIG. 18 is a perspective view illustrating an appearance of a movable lens unit according to the fifth modified example, and FIG. 19 is a sectional view illustrating the movable lens unit according to the fifth modified example.

As illustrated in FIGS. 18 and 19, the movable lens unit 40 may include one permanent magnet 55 having a substantially semicircular cross-section and stacked from the upper side of the first coil 44 and the second coil 45.

Other configurations are the same as those in the above-described embodiment. Also such a configuration enables further size reduction of the movable lens unit 40.

Note that a flexible endoscope is exemplified in the present embodiment. However the present invention is not limited to such a flexible endoscope but is a technique also applicable to rigid endoscopes for surgery and industrial endoscopes.

The invention recited in the above-described embodiment is not limited to the embodiment as described above, and various modifications are possible in the practical stages within a range without departing from the gist of the invention. Furthermore, the above embodiment includes inventions at various stages, and various inventions can be extracted by appropriately combining a plurality of disclosed components.

For example, even when some of the components are removed from all the components shown in the above embodiment, a configuration from which the components are eliminated can be extracted as an invention in the case where the configuration can solve the above-described problem and attain the above-described effects of the invention.

With the present invention, it is possible to provide a linear actuator for endoscope, an optical unit for endoscope, and an endoscope, the lengths of which can be reduced and assembling performance is improved compared with the conventional ones.

What is claimed is:

1. A linear actuator for endoscope, the linear actuator comprising:
a movable frame made of a magnetic material, the movable frame holding a lens;
a fixed frame in which the movable frame is provided so as to be movable forward and backward, the fixed frame being made of a non-magnetic material;
two coils formed by being wound on an outer surface of the fixed frame, the two coils being disposed side by side in a longitudinal axis direction;
a permanent magnet disposed in a manner stacked and superimposed on the two coils; and
two yokes respectively disposed at a front and a back of the permanent magnet and the two coils.

2. The linear actuator for endoscope according to claim 1, wherein the movable frame holds a movable lens that configures an optical system.

3. The linear actuator for endoscope according to claim 1, wherein the two coils are wound on the fixed frame so as to have a same winding direction, and provided in different circuits to which current is supplied independently.

4. The linear actuator for endoscope according to claim 1, wherein the two coils are wound on the fixed frame so as to have winding directions opposite to each other, and provided in a serial circuit.

5. The linear actuator for endoscope according to claim 1, wherein the permanent magnet has a cylindrical shape and is disposed so as to cover entire circumferences of the two coils.

6. The linear actuator for endoscope according to claim 1,
wherein the permanent magnet comprises two permanent magnets,
wherein the two permanent magnets are disposed at positions symmetrical to an optical axis of the lens.

7. An optical unit for endoscope comprising:
the linear actuator for endoscope according to claim 1.

8. An endoscope comprising:
an insertion portion including a distal end portion in which the optical unit for endoscope according to claim 7 is disposed.
